(19) United States
(12) Patent Application Publication
(10) Pub. No.: US 2017/0143386 A1
(43) Pub. Date: May 25, 2017

(54) Nail System and Method for an Olecranon Osteotomy

(71) Applicant: DePuy Synthes Products, Inc., Raynham, MA (US)

(72) Inventors: George A. MIKHAIL, West Chester, PA (US); Glen PIERSON, West Chester, PA (US)

(21) Appl. No.: 15/401,834

(22) Filed: Jan. 9, 2017

Related U.S. Application Data
(63) Continuation of application No. 11/746,175, filed on May 9, 2007, now abandoned.
(60) Provisional application No. 60/799,428, filed on May 9, 2006.

(51) Int. Cl. A61B 17/72; A61B 17/17

(52) U.S. Cl. CPC A61B 17/72; A61B 17/1725; A61B 17/1739; A61B 17/7225

(57) ABSTRACT

The present invention relates to a nail system for fixation of an olecranon osteotomy or fracture. The nail system of the present invention is securely held in place in the intramedullary canal to ensure proper anatomic reduction of an osteotomy. Further, the invention provides an insertion device and a method for inserting the osteotomy nail system, securing the osteotomy nail system, and fixing the osteotomy.

### RELATED APPLICATIONS

This application claims benefit of U.S. Application Ser. No. 60/799,428 filed May 9, 2006, the contents of which are incorporated herein by reference.

### TECHNOLOGY FIELD

The invention generally relates to a device for fixation of a condyle. More particularly, the invention relates to a nail for fixation of an olecranon osteotomy or fracture. The invention further relates to an instrument and a method for securing the nail.

### BACKGROUND

Distal humerus fractures in adults are optimally treated with open anatomic reduction and stable fixation to allow early anatomic restoration and upper extremity range of motion. Therefore, surgical exposure is often necessary to treat fractures of the distal humerus. The olecranon osteotomy provides wide exposure of the distal humerus, which exposure is optimal for repairing complex distal humerus fractures. The olecranon osteotomy is a surgical procedure in which the large process on the upper end of the ulna is cut to enable proximal retraction of the triceps and exposure of the distal humerus. The olecranon osteotomy, however, is ancillary to the repair of a distal humerus fracture and results in additional surgical time. As a result, repair of olecranon osteotomies are sometimes rushed, resulting in unsatisfactory reduction and articular function.

The techniques most frequently utilized for securing olecranon osteotomies are fixation using a tension band wire technique and K-wires, plate fixation, and fixation using an intramedullary lag screw with a tension band wire. These techniques, however, suffer from complications. Fixation using tension band wire and K-wires may result in K-wire and wire prominence under the skin causing local irritation and requiring a secondary operation for removal. Similarly, plate fixation may also result in plate prominence under the skin causing local irritation and requiring removal. Fixation with a lag screw is problematic, because the curvature of the proximal ulna may make accurate placement of the screw down the intramedullary canal difficult. Alternatively, the lag screw may be used with tension band wiring to address this problem, but use of tension band wiring has the associated problems mentioned above.

In light of the problems and disadvantages related to the existing techniques, a new technique is needed to more effectively and efficiently repair olecranon osteotomies.

### SUMMARY

The invention provides an osteotomy nail system that is securely held in place in the intramedullary canal and that ensures proper anatomic reduction of an osteotomy or a fracture. Further, the invention provides an insertion device and a method for inserting the osteotomy nail system, securing the osteotomy nail system, and fixing the osteotomy.

According to one embodiment, the osteotomy nail system comprises an osteotomy nail having a body portion and a threaded head portion, a nail cap, and at least two screws. The body portion of the osteotomy nail may include an upper screw bore, an intermediate alignment hole, and a lower screw bore. The screw bores are preferably positioned oblique to the longitudinal axis of the osteotomy nail and the alignment hole is preferably positioned transverse to the longitudinal axis of the osteotomy nail. The nail cap has a distal portion and a proximal portion. The distal portion of the nail cap may be threaded such that the nail cap can be screwed onto the threaded head portion of the osteotomy nail and provide compression.

According to another aspect of the invention, an insertion device is used to properly align and insert the osteotomy nail into the intramedullary canal. In one embodiment, the insertion device comprises an aiming arm, an inserter tool, an inserter cap, an alignment tool, and a clamping fastener.

In one embodiment of the insertion device, the aiming arm has an arm portion connected to a body portion. The body portion includes an upper screw channel, an intermediate alignment channel, and a lower screw channel to complement the bores and hole in the osteotomy nail. The arm portion has a collar hole and a slit through its body to provide a means for clamping the osteotomy nail into correct orientation relative to the aiming arm prior to insertion into a bone. The slit extends through the arm portion and radially from the collar hole to the free end of the arm portion on a plane coextending from the central axis of the collar hole. Further, the arm portion has a clamping hole that extends transverse to the plane of the slit. The holes and channels of the aiming arm are arranged such that: the central axes of the screw channels intersect the central axis of the collar hole at angles corresponding to the angles at which the screw bores in the osteotomy nail intersect the longitudinal axis of the osteotomy nail; and the central axis of the alignment channel intersects the central axis of the collar hole at the same angle and position that the central axis of the alignment hole of the osteotomy nail intersects the longitudinal axis of the osteotomy nail.

In one embodiment of the insertion device, the inserter tool has a distal end, a proximal end, an annular flange between the two ends, and an inserter cap. The distal end is threaded such that the inserter tool can be secured onto the threaded head portion of the osteotomy nail. The proximal end is also threaded on its free end. The proximal end of the inserter tool is inserted through the collar hole of the aiming such that the threads of the proximal end extend outside the collar hole.

In one embodiment of the insertion device, the inserter cap has a distal end and a proximal end. The distal end is threaded such that the inserter cap can be secured onto the threaded portion of the proximal end of the inserter tool. When the inserter cap is secured to the proximal end of the inserter tool, the inserter tool can be axially secured within the collar hole of the aiming arm. The inserter tool, however, may be free to rotate around its longitudinal axis.

In another embodiment of the insertion device, the inserter tool has a distal end, a proximal end, and an annular flange disposed around the proximal end. The distal end is threaded such that the inserter tool can be secured onto the threaded head portion of the osteotomy nail. Thus, the distal end of the inserter tool may be attached to the osteotomy nail and inserted through the collar hole of the aiming such that the annular flange of the proximal end stops the inserter tool from falling through the collar hole.

In one embodiment of the insertion device, the alignment tool is a rod that is sized such that it may be snugly inserted through the alignment channel of the aiming arm and into the alignment hole of the osteotomy nail. The alignment tools aligns the alignment channel of the aiming arm and the alignment hole of the osteotomy nail such that the corresponding screw channels of the aiming arm and screw bores of the osteotomy nail are also aligned.

In one embodiment of the insertion device, the aiming arm fastener is a bolt that is inserted through the clamping hole of the aiming arm such that the collar hole and slit of the aiming arm may be tightened. Tightening of the collar hole and slit of the aiming arm locks the combination of the inserter tool and osteotomy nail with respect to the aiming arm by applying radial pressure to the proximal end of the inserter tool. Therefore, once the alignment channel on the aiming arm and the alignment hole in the osteotomy nail have been aligned, the alignment may be maintained by tightening the collar hole and slit of the aiming arm with the clamping fastener. Alternatively, a cam lock may be used to secure the inserter tool in the collar hole of the aiming arm.

According to another aspect of the invention, the aiming arm may be used to insert the osteotomy nail into the upper end of the ulna to enable repair of an olecranon osteotomy or olecranon fracture. According to a preferred method, the osteotomy nail is inserted and secured in the upper end of the ulna before an olecranon osteotomy is performed.

According to one method of performing an olecranon osteotomy, a hole is drilled through the upper end of the ulna into the intramedullary canal to a depth adequate to accommodate the osteotomy nail below the site of the osteotomy. The threaded head portion of the osteotomy nail is secured to the threaded distal end of the inserter tool of the insertion device. The proximal end of the inserter tool is inserted through the collar hole of the aiming arm of the insertion device. The inserter cap is secured onto the proximal end of the inserter tool such that the inserter tool is captured in the collar hole of the aiming arm. The alignment tool is inserted through the alignment channel of the aiming arm and the osteotomy is adjusted rotationally and axially to allow the alignment tool to be inserted into the alignment hole of the osteotomy nail. Once the alignment tool is inserted through the alignment channel of the aiming arm and into the alignment hole of the osteotomy nail, the clamping fastener may be inserted through the clamping hole to tighten the collar hole and slit of the aiming arm and maintain the alignment of the aiming arm with respect to the osteotomy nail. The alignment tool can be removed and the osteotomy nail may be inserted into the predrilled hole in the upper end of the ulna using the insertion device.

The aiming arm of the insertion device is disposed outside the bone with its screw channels aligned with the corresponding screw bores of the osteotomy nail disposed within the intramedullary canal of the bone. The alignment of the screw channels in the aiming arm and the screw bores in the osteotomy nail allow the osteotomy nail to be properly secured in the intramedullary canal of the bone. First, a drill is passed through a screw channel in the aiming arm, through one side of the bone, through a corresponding screw bore in the osteotomy nail, and through the other side of the bone. Preferably, a drill sleeve and a protection sleeve are disposed in the screw channel. The drill sleeve is disposed inside the protection sleeve, such that the drill is inserted through the drill sleeve and into the bone without causing damage to the surrounding soft tissue. Second, the osteotomy nail is secured in the intramedullary canal of the bone by inserting a screw through the screw channel in the aiming arm, into one side of the bone, into the corresponding screw bore in the osteotomy nail; and into the other side of the bone. The drilling and screwing process is repeated for the other screw channels and screw bores, thereby securing the osteotomy nail in the intramedullary canal of the bone.

The screw bores in the osteotomy nail are preferably set at opposing angles oblique to the longitudinal axis of the osteotomy nail to limit the amount of longitudinal and rotational movement of the osteotomy nail while in the bone. Further, the screws preferably have threaded heads that contact the body portion of the osteotomy nail to limit lateral and rotational movement of the osteotomy nail in the bone due to clearance between the screws and the screw bores. The preferred combination of having screw bores at opposing angles and screws with threaded heads that contact the body portion of the osteotomy nail limits longitudinal, lateral, and rotational movement of the osteotomy nail in the bone so that an osteotomy may be properly realigned and reattached.

Once the osteotomy nail is secured in the intramedullary canal of the ulna, the insertion device may be disassembled and detached from the osteotomy nail by, for example: removing the inserter cap from the inserter tool, releasing the clamping fastener, removing the aiming arm from inserter tool, and removing the tip of the inserter tool from the osteotomy nail. Then, an olecranon osteotomy may be performed above the location of the osteotomy nail. Thereafter, the sectioned portion of the bone may be replaced and the nail cap may be inserted through the hole in the sectioned portion of the bone and secured to the head portion of the osteotomy nail. The nail cap provides compression and allows proper reduction of the olecranon osteotomy.

According to another method, the osteotomy nail may be inserted and secured in the ulna in the manner described above to reduce an olecranon fracture instead of an olecranon osteotomy.

According to one method, the osteotomy nail is removed using the insertion device and an alignment drill. The alignment drill is used to drill through bone that may grow over an implanted osteotomy nail and to locate the alignment hole of the osteotomy nail. Once the alignment hole of the nail is located with the alignment drill, the alignment drill is at least partially inserted into the alignment hole of the osteotomy nail. Then, the nail cap is removed from the osteotomy nail and the aiming arm is positioned over the alignment drill such that the alignment drill is disposed through the alignment channel of the aiming arm. Thus, the alignment drill aligns the alignment channel of the aiming arm with the alignment hole of the osteotomy nail. Then, the inserter tool is inserted through the collar hole of the aiming arm such that the distal end of the inserter tool may is attached to the head portion of the osteotomy nail.

In accordance with this method, once the alignment drill is placed through the alignment channel of the aiming arm and the inserter tool is attached to the osteotomy nail through the collar hole of the aiming arm, the screw channels of the aiming arm and the screw bores of the osteotomy nail are aligned. Then, the alignment drill may be removed from the aiming arm and a drill may be inserted through the screw channels of the aiming arm to drill through the bone and locate the heads of the screws that secure the osteotomy nail in the bone. After the drill is removed, a screwdriver may be inserted through the screw channels of the aiming arm to remove the screws from the osteotomy nail. Thus, once the screws are removed, the osteotomy nail can be removed from the bone by pulling the inserter tool attached to the nail.

Additional features and advantages of the invention will be made apparent from the following detailed description of illustrative embodiments that proceeds with reference to the accompanying drawings.

### BRIEF DESCRIPTION OF THE DRAWINGS

The foregoing summary, as well as the following detailed description of the preferred embodiments, is better understood when read in conjunction with the appended drawings. For the purpose of illustrating the invention, there are shown in the drawings embodiments that are presently preferred, it being understood, however, that the invention is not limited to the specific methods and instrumentalities disclosed. In the drawings:

FIG. 1A shows a view of an exemplary osteotomy nail system secured in a bone.

FIG. 1B shows a top view of an exemplary osteotomy nail system.

FIG. 1C shows a side view of the osteotomy nail shown in FIG. 1B.

FIG. 1D shows a top view of an exemplary osteotomy nail system.

FIG. 1E shows a side view of the osteotomy nail system shown in FIG. 1D.

FIG. 2A shows an AP view of an exemplary osteotomy nail.

FIG. 2B shows a cross sectional view of the osteotomy nail shown in FIG. 2A taken along sectional line A-A of FIG. 2A.

FIG. 3A shows a lateral view of an exemplary screw.

FIG. 3B shows a cross sectional view of the screw shown in FIG. 3A taken along sectional line B-B of FIG. 3A.

FIG. 4A shows a lateral view of an exemplary nail cap.

FIG. 4B shows a cross sectional view of the nail cap shown in FIG. 4A taken along sectional line C-C of FIG. 4A.

FIG. 4C shows the distal portion of an exemplary nail cap attached to the head portion of an exemplary osteotomy nail.

FIG. 5 shows an assembled exemplary insertion device.

FIG. 6A shows a lateral view of an exemplary aiming arm.

FIG. 6B shows a back view of the aiming arm shown in FIG. 6A.

FIG. 6C shows a top view of the aiming arm shown in FIG. 6A.

FIG. 6D shows a cross sectional view of the aiming arm shown in FIG. 6A taken along sectional line D-D of FIG. 6A.

FIG. 6E shows a cross sectional view of the aiming arm shown in FIG. 6A taken along sectional line E-E of FIG. 6B.

FIG. 7A shows a lateral view of an exemplary inserter tool.

FIG. 7B shows a cross sectional view of the inserter tool shown in FIG. 7A taken along sectional line F-F of FIG. 7A.

FIG. 7C shows a top view of the proximal end of the inserter tool shown in FIG. 7A.

FIG. 7D shows a top view of the proximal end of another exemplary inserter tool.

FIG. 8A shows a top view of an exemplary inserter cap.

FIG. 8B shows a cross sectional view of the inserter cap shown in FIG. 8A taken along sectional line H-H of FIG. 8A.

FIG. 9A shows a lateral view of an exemplary inserter collet and collet cap.

FIG. 9B shows a top view of the inserter collet shown in FIG. 9A.

FIG. 9C shows a top view of another exemplary inserter collet.

FIG. 9D shows a cross sectional view of the inserter collet and collet cap shown in FIG. 9A taken along sectional line I-I of FIG. 9A.

FIG. 9E shows a cross sectional view of the inserter collet of FIG. 9A with the collet cap secured thereon.

FIG. 10A shows a lateral view of another exemplary inserter tool.

FIG. 10B shows a top view of the proximal end of the inserter tool shown in FIG. 10A.

FIG. 11 shows a lateral view of an exemplary alignment tool.

FIG. 12A shows a lateral view of an exemplary clamping fastener.

FIG. 12B shows a cross sectional view of the clamping fastener shown in FIG. 11A taken along sectional line J-J of FIG. 11A.

FIG. 13A shows a view of an assembled exemplary insertion device holding an exemplary osteotomy nail with the inserter tool shown in FIGS. 7A-7D.

FIG. 13B shows a view of an assembled exemplary insertion device holding an exemplary osteotomy nail with the inserter tool shown in FIGS. 10A-10B.

FIG. 14 shows an exemplary opening drilling operation for inserting an osteotomy nail into the intramedullary canal of a bone.

FIG. 15 shows an exemplary drilling operation for securing the osteotomy nail into the intramedullary canal of a bone.

FIG. 16 shows an exemplary screwing operation for securing the osteotomy nail into the intramedullary canal of a bone.

FIG. 17A shows an exemplary osteotomy nail secured in the intramedullary canal of a bone below the site of an osteotomy.

FIG. 17B shows an exemplary nail cap secured on the osteotomy nail shown in FIG. 17A to reduce the osteotomy.

FIG. 18A shows an exemplary nail system implanted in a bone.

FIG. 18B shows an exemplary alignment drill used to locate the alignment hole of the osteotomy nail shown in FIG. 18A.

FIG. 18C shows an exemplary aiming arm with its alignment channel disposed over the alignment drill shown in FIG. 18B.

FIG. 18D shows an exemplary inserter tool being inserted through collar hole of the aiming arm and attached to the osteotomy nail shown in FIG. 18C.

FIG. 18E shows an exemplary drill inserted through a screw channel of the aiming arm to locate a screw in the osteotomy nail shown in FIG. 18D.

### DETAILED DESCRIPTION OF ILLUSTRATIVE EMBODIMENTS

The present invention concerns an osteotomy nailing system. The osteotomy nailing system comprises an osteotomy nail system 10 and an insertion device 30. The osteotomy nail system 10 ensures proper anatomic reduction of an osteotomy. The insertion device 30 allows the osteotomy nail system 10 to be securely fixed in the intramedullary canal of a bone. Further, the invention provides a method for inserting the osteotomy nail system 10, securing the osteotomy nail system 10, and fixing an osteotomy using the insertion device 30.

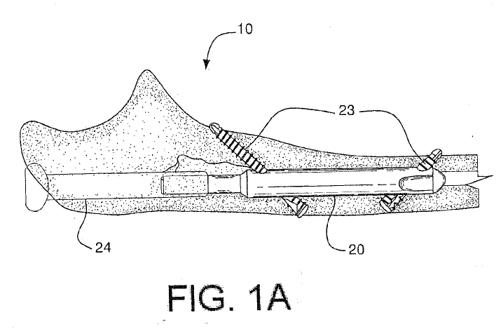

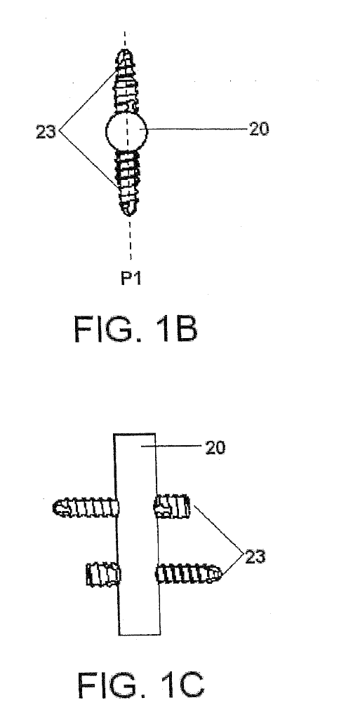

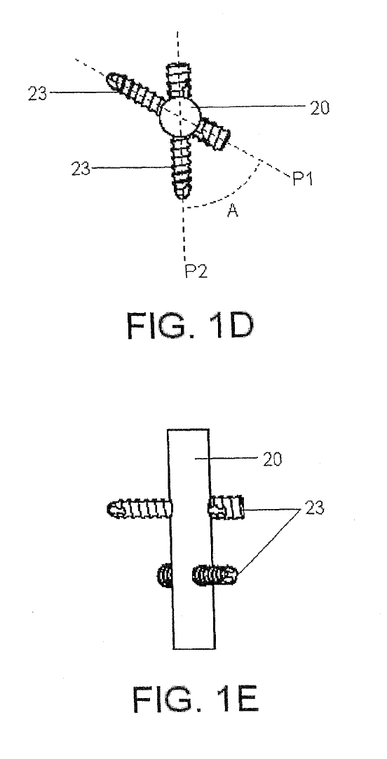

In one embodiment, as shown in FIG. 1A, the osteotomy nail system 10 comprises an osteotomy nail 20, at least two screws 23, and a nail cap 24. As shown, the osteotomy nail 20 is fixed in a bone with the screws 23 and nail cap 24.

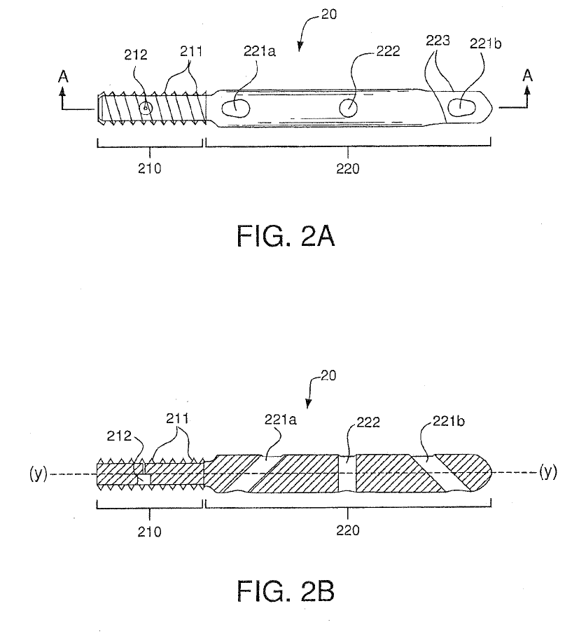

Referring to FIGS. 2A-2B, according to one embodiment, the osteotomy nail 20 comprises a head portion 210 and a body portion 220 that are preferably integrally formed into a rod-like shape having a longitudinal axis (y). Although the osteotomy nail 20 is shown having a generally round cylindrical shape, other shapes (e.g. polygonal cylinder) may be used as well. The head portion 210 has fastening means 211 disposed thereon. As shown in FIG. 2A, the fastening means is preferably a threaded section, but may include any other suitable fastening means, such as a snap-on or clip-on structure. In the embodiment shown in FIG. 2A, the fastening means 211—shown as a threaded section—is disposed on the surface of at least a portion of the head portion 210, but may be alternatively disposed in an interior surface of a recess (not shown) at the tip of the head portion 210 of the osteotomy nail 20. In an embodiment having a threaded section as fastening means 211, as shown in FIG. 2A, the head portion 210 may further include securing means for preventing objects secured by the threads from inadvertently reversing off the threads. For example, such securing means may include a patch 212 made of deformable material disposed on the head portion 210 of the nail 20 as shown in FIG. 2A.

As shown in the preferred embodiment depicted in FIG. 2A-2B, the body portion 220 of the osteotomy nail 20 includes at least two screw bores 221 (an upper screw bore 221a and a lower screw bore 221b), an alignment hole 222, and at least two planar surfaces 223. As shown in FIG. 2B, the screw bores 221 are preferably disposed oblique to the longitudinal axis (y) of the osteotomy nail 20 at angles opposing each other. It is preferred that the screw bores 221 be disposed at opposing angles between about 30° and 60° from the longitudinal axis (y). As shown in FIGS. 1B and 1C, the screw bores 221 are preferably disposed on the same plane Pl. The screw bores, however, may be disposed on independent planes that coextend from the longitudinal axis (y) of the osteotomy nail 20 and that are angularly spaced apart. For example, as shown in FIGS. 1D and 1E, the screw bores may be disposed on independent planes P1, P2 that are spaced apart by an angle A of 0° to 90°.

The alignment hole 222, as shown in FIG. 2B, is preferably positioned perpendicular to the longitudinal axis (y) of the osteotomy nail 20, but the alignment hole 222 may be positioned oblique to the longitudinal axis (y) of the osteotomy nail 20. Also, as shown in FIG. 2B, the alignment hole 222 extends through the body portion 220 of the osteotomy nail 20, but the alignment hole 222 may only partially extend into the body portion 220 of the osteotomy nail 20. As shown in FIG. 2A, the planar surfaces 223 are disposed on opposite sides the distal end of the body portion 220 of the osteotomy nail 20.

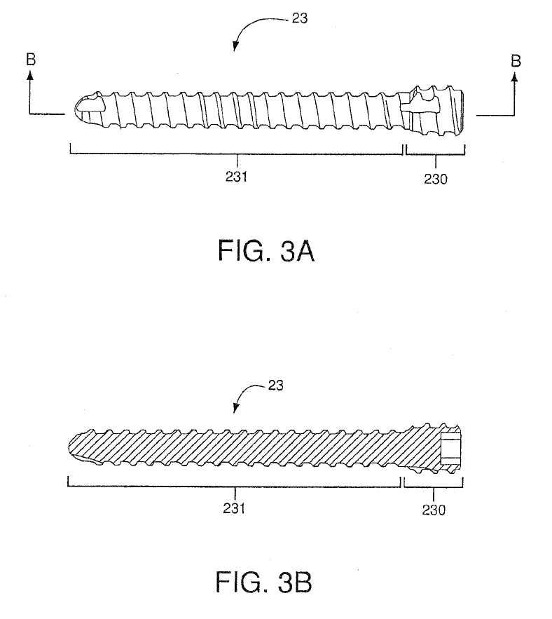

In one embodiment, as shown in FIGS. 3A-3B, the screws 23 preferably comprise a head portion 230 having a diameter larger than the diameter of a body portion 231. Also, the screws 23 are preferably threaded throughout the length of both the body 231 and head 230 portions.

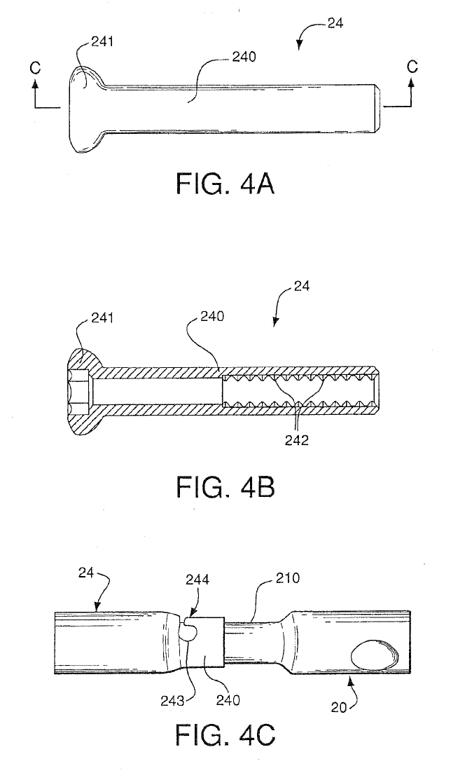

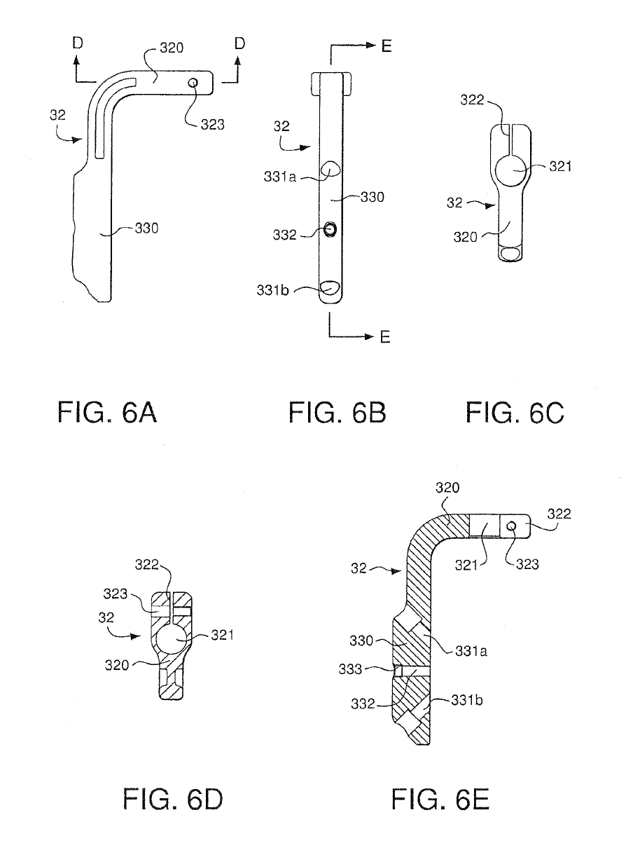

As shown in FIGS. 4A-4B, the nail cap 24 preferably comprises a distal portion 240 having a diameter smaller than the diameter of a proximal portion 241. Also, the distal portion 240 includes fastening means 242, such as, for example, a threaded section as shown in FIG. 4B that complements the fastening means 211 of the head portion 210 of the osteotomy nail 20. The fastening means 211 may, however, include any other suitable means, such as a snap-on or clip-on structure. In the embodiment shown in FIG. 4B, the fastening means 242—shown as a threaded section—are disposed on the interior surface of a recess in the distal portion 240 of the nail cap 24, but may be disposed on an exterior surface of the distal portion of the nail cap 24. Additionally, as shown in FIG. 4C, the nail cap 24 may include securing means in the form of a cut 243 in the distal portion 240 that forms a cantilever 244. The cantilever 244 is bent radially inward such that the cantilever 244 interferes with the fastening means 211 of the osteotomy nail 20 and creates a frictional hold between the nail cap 24 and osteotomy nail 20. The frictional hold of the cantilever 244 prevents the nail cap 24 from reversing off of the osteotomy nail 20.

Referring back to FIGS. 1A-4B, the osteotomy nail 20 is preferably secured in a bone by driving the screws 23 through the screw bores 221 of the body portion 220 until the head portion 230 of the screw 23 contacts the body portion 220 of the osteotomy nail 20. As shown in FIG. 1A the screws 23 may be inserted into the osteotomy nail 20 in the same direction, but as shown in FIGS. 1B and 1C, the screws 23 may be inserted into the osteotomy nail 20 in opposing directions. The threads on the head portions 230 of the screws 23 allow the head portions 230 of the screws 23 to be driven into the bone and into contact with the body portion 220 of the osteotomy nail 20. By providing threads on the head portions 230 of the screws 23 and driving the head portions 230 of the screws 23 into contact with the body portion 220 of the osteotomy nail 20, play between the screws 23 and the nail 20 may be limited and the nail 20 may be more stably fixed in the bone. Further, the threads on the head portions 230 of the screws 23 allow the head portions 230 of the screws 23 to be driven into the bone so that the head portions 230 of the screws 23 are substantially flush with the bone. The screw bores 221 in conjunction with the screws 23 provide a means for securing the osteotomy nail 20 in a bone. It is contemplated, however, that the means for securing the osteotomy nail 20 in a bone may comprise other suitable structures or additional structures.

Again, referring to FIGS. 1-4B, the nail cap 24 is secured on the head portion 210 of the osteotomy nail 20 by mating the complementary fastening means on the distal portion 240 of the nail cap 24 and the head portion 210 of the osteotomy nail 20. In the embodiment of FIG. 1A, the nail cap 24 and the head portion 210 of the osteotomy nail 20 are secured together by mating threads on an interior surface of a recess in the distal portion 240 of the nail cap 24 and threads on the surface of the head portion 210 of the osteotomy nail 20. As described above, in one embodiment, the head portion 210 of the osteotomy nail 20 includes securing means, such as a patch 212 made of deformable material, that prevent the nail cap 24 secured by the threads 211, 242 from inadvertently reversing off the threads. In another embodiment, the securing means may be the cantilever 244 of the nail cap 24 that interferes with the threads 211 of the osteotomy nail 20 and prevents the nail cap 24 from reversing off the threads 211.

The fastening means 242, however, may be disposed on the exterior surface of the of the distal portion 240 of the nail cap 24 and the fastening means 211 may be disposed on the interior surface of a recess in the head portion 210 of the osteotomy nail 20. In such an embodiment, the distal portion 240 of the nail cap 24 would be inserted into the recess in the head portion 210 of the osteotomy nail 20 and fastened to the osteotomy nail 20.

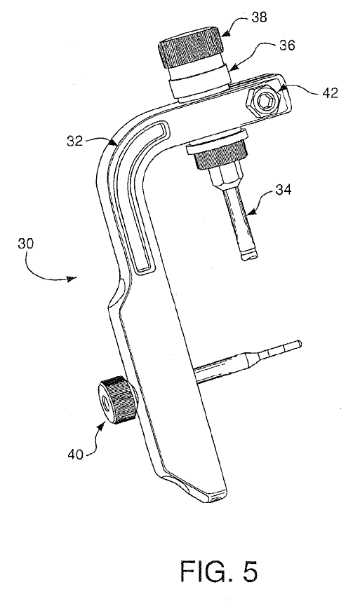

Now referring to FIG. 5, an embodiment of the insertion device 30 is shown. As shown, the insertion device 30 may comprise an aiming arm 32, an inserter tool 34, inserter collet 36, an inserter cap 38, an alignment tool 40, and a clamping fastener 42. In general, the insertion device 30 is used to properly align the osteotomy nail 20 for positioning within the intramedullary canal of a bone so that the screws 23 can be inserted into the screw bores 221 to secure the osteotomy nail 20 in a bone.

As shown in FIGS. 6A-6E, the aiming arm 32 comprises an arm portion 320 connected to a body portion 330. In the embodiment shown by FIGS. 6A-6E, the arm portion 320 and body portion 330 form an L-shaped aiming arm 32, but they may connect to form any other suitable shape that conforms to the description below.

As depicted and as preferred, the body portion 330 includes two screw channels 331 (an upper screw channel 331a and a lower screw channel 331b) and an alignment channel 332 to complement the screw bores 221 (upper screw bore 221a and lower screw bore 221b) and alignment hole 222 in the osteotomy nail 20. As shown in FIG. 6E, the screw channels 331 and alignment channel 332 extend through the body portion 330. The arm portion 320 includes a collar hole 321, and may further include a slit 322 and a clamping hole 323. As shown in FIGS. 6A-6E, the screw channels 331, alignment channel 332, and collar hole 321 of the aiming arm 32 are arranged such that:
the central axes of the screw channels 331 intersect the central axis of the collar hole 321 at angles corresponding to the angles at which the screw bores 221 in the osteotomy nail 20 intersect the longitudinal axis of the osteotomy nail 20; and
the central axis of the alignment channel 332 intersects the central axis of the collar hole 321 at the same angle and position that the central axis of the alignment hole 222 of the osteotomy nail 20 intersects the longitudinal axis of the osteotomy nail 20.

In one embodiment, as shown in FIGS. 6A-6E, a slit 322 extends through the arm portion 320 and extends radially from the collar hole 321 to the free end of the arm portion 320 on a plane coextending from the central axis of the collar hole 321—dividing the arm portion 320 into two sections. Further, a clamping hole 323 extends transverse to the plane of the slit 322. Also, as shown in FIG. 6E, there is a fastening means 333 disposed inside the alignment channel 332 of the body portion 330 of the aiming arm 32. The fastening means can be any suitable means, such as, for example, threads, a snap-on structure, a clip-on structure, or a cam lock.

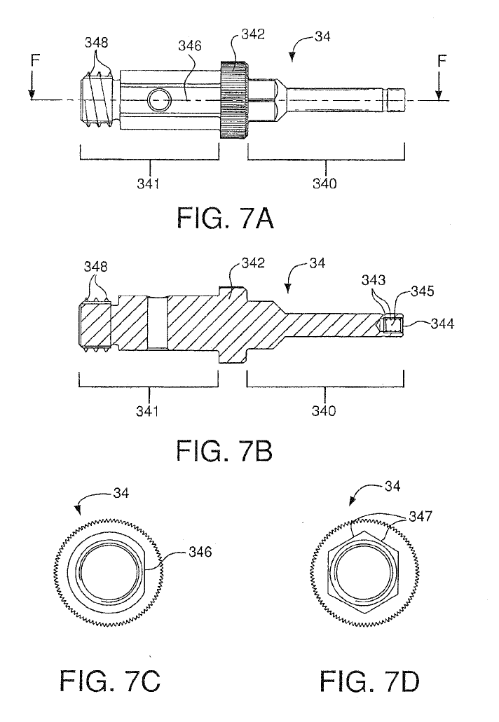

In the embodiment shown in FIGS. 7A-7D, the inserter tool 34 has a distal portion 340, a proximal portion 341, and an annular flange 342 between the two portions 340, 341. The inserter tool 34 is used to hold the osteotomy nail 20 by engaging the head portion 230 of the nail 20 with its distal portion 340. In FIG. 7B, the distal portion 340 has fastening means 343—shown as a threaded section—on an interior surface 344 of a recess 345 in the tip of the distal portion 340. However, the fastening means 343 need not be a threaded section and can be any other suitable means that complement the fastening means 211 on the head portion 210 of the osteotomy nail 20 such that the inserter tool 34 can be secured to the osteotomy nail 20. Further, the fastening means 343 need not be disposed in a recess 345 and can be disposed on the outer surface of the distal portion 340 of the inserter tool 34. Also, as shown in FIGS. 7C-7D, the proximal portion 341 preferably has a cylindrical structure with at least one planar surface 346 along its longitudinal axis, or a polygonal cylindrical structure 347. The proximal portion 341 may also have a round cylindrical structure (not shown). Further, as shown in FIGS. 7A-7B, there are fastening means 348—shown as a threaded section—disposed adjacent to the tip of the proximal portion 341 of the inserter tool 34. The fastening means 348, however, need not be a threaded section and can be any other suitable means. Further, for example, the fastening means 348 may be disposed in a recess (not shown) in the proximal portion 340 of the inserter tool 34.

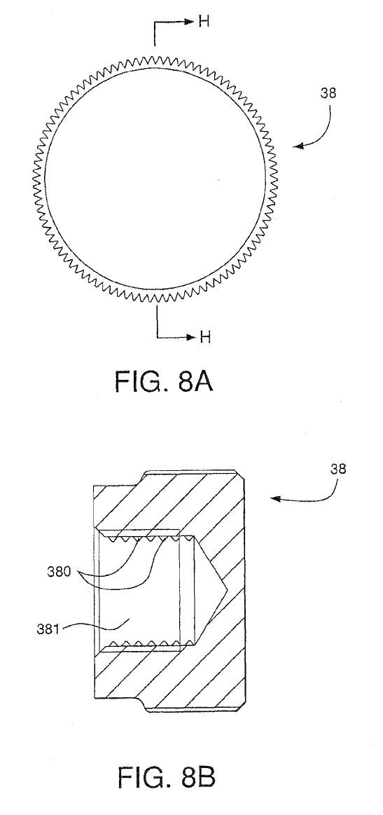

In the embodiment shown in FIGS. 8A-8B, the inserter cap 38 is a round cylindrical sleeve having fastening means 380 disposed on the surface of the interior 381 of the inserter cap. The inserter cap 38 is used to cap the proximal portion 340 of inserter tool 34. At least a portion of the inserter cap 38 has a diameter greater than the diameter of the proximal end 341 of the inserter tool 34. The fastening means 348 is shown as a threaded section, but it can be any other suitable fastening means. This particular embodiment is an inserter cap 38 having fastening means 380 on the interior 381 that complement an inserter tool 34, embodied in FIGS. 7A-7D, having fastening means 348 on an outer surface of the proximal portion 341 of the inserter tool 34. As described above, however, the fastening means 348 may be disposed in a recessed surface. Therefore, the inserter cap 38 may also be a bolt having fastening means 380 on an outer surface to complement the fastening means 348 disposed in a recess in the proximal portion 340 of the inserter tool 34.

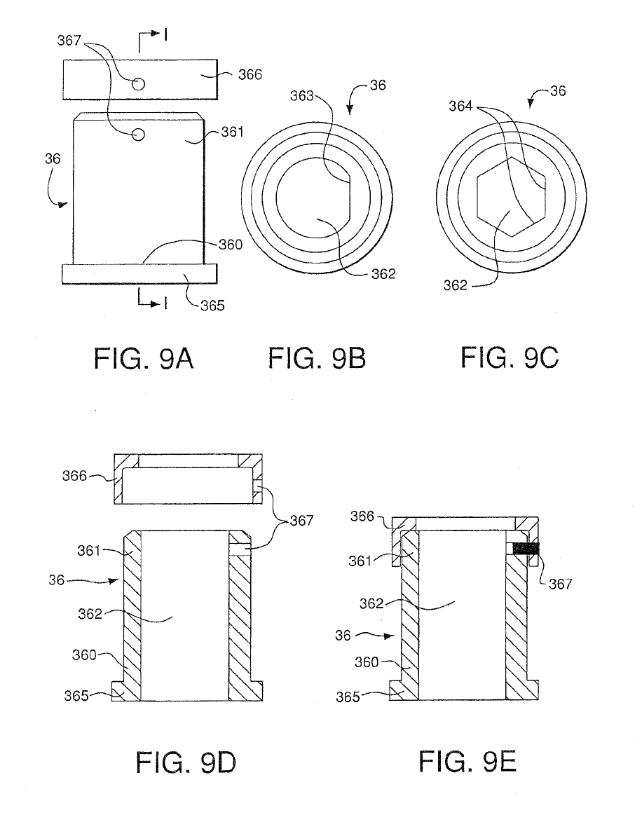

As shown in FIGS. 9A-9E, the inserter collet 36 is a substantially cylindrical sleeve having a bottom end 360, a top end 361, and an interior 362. The inserter collet 36 is used to surround at least part of the proximal portion 341 of the inserter tool 34. Preferably, the interior 362 has at least one planar surface 363 corresponding to a planar surface 346 of the proximal portion 341 of the inserter tool 34 or, alternatively, a polygonal structure 364 corresponding to a polygonal structure 347 of the proximal portion 341 of the inserter tool 34. Also, as shown in FIGS. 9A and 9D-9E, the inserter collet 36 may include an annular flange 365 disposed around the bottom end 360 of the inserter collet and a collet cap 366 that may be secured around the top end 361 of the inserter collet 36. The inserter collet 36 may have fastening means 367 for attaching the collet cap 366 to the top end 361 of the inserter collet 36. As shown in FIGS. 9A and 9D-9E, the fastening means may include complementary holes in the collet cap 366 and the top end 361 of the inserter collet 36 that receive a pin to lock the collet cap 366 to the inserter collet 36. In another embodiment, not shown, the inserter collet 36 may comprise a sleeve without an annular flange 365 or a collet cap 366.

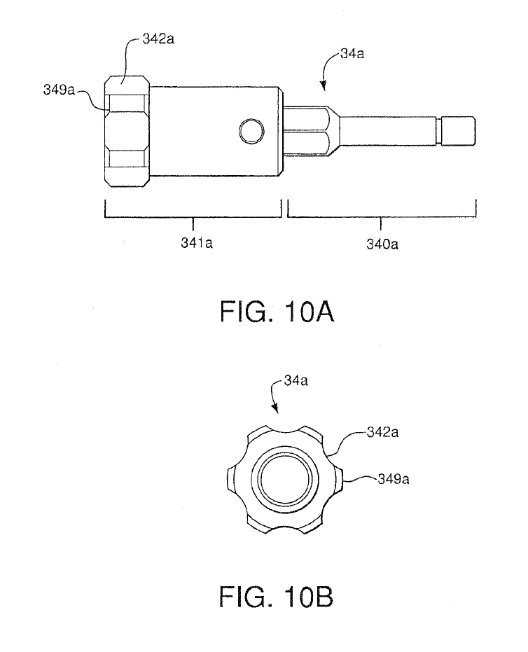

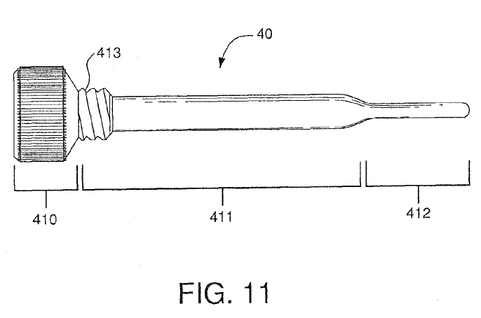

In another embodiment, the inserter tool 34 of the insertion device 30 does not use an inserter collet 36, a collet cap 366, or an inserter cap 38. Rather, as shown in FIG. 10A, the inserter tool 34a has a distal portion 340a, a proximal portion 341a, and an annular flange 342a disposed around the proximal portion 341a. As shown, the annular flange 342a is preferably disposed around the proximal end of the proximal portion 341a of the inserter tool 34a. Also, the annular flange 342a may have notches 349a disposed around its circumference so that the inserter tool 34a can be better gripped and rotated about its longitudinal axis. Like in the embodiment of FIGS. 7A and 7B, the distal end 340a has fastening means 343 (not shown) so that the inserter tool 34a can be secured onto the head portion 210 of the osteotomy nail 20.

The alignment tool 40 is used to align the alignment channel 332 of the aiming arm 32 with the alignment hole 222 of the osteotomy nail 20. In one embodiment, as shown in FIG. 11, the alignment tool 40 has a head 410, a body 411, a tip 412, and fastening means 413. The body 411 has a diameter that is slightly smaller than the diameter of the alignment channel 332 of the aiming arm 32, and the tip 412 has a diameter that is slightly smaller than the diameter of the alignment hole 222 of the osteotomy nail 20 such that alignment tool 40 may be snugly inserted through the alignment channel 332 of the aiming arm 32 and into the alignment hole 222 of the osteotomy nail 20. The alignment tool 40, however, may have any other suitable structure that allows it to pass through the alignment channel 332 of the aiming arm 32 and into the alignment hole 222 of the osteotomy nail 20. For example, the alignment tool 40 may be a rod. In the embodiment shown in FIG. 11, the fastening means 413—shown as a threaded section—is disposed on the surface of at least a portion of the body 411 adjacent to the head 410 of the alignment tool 40. The fastening means 413 on the alignment tool 40 complement the fastening means 333 in the alignment channel 332 of the aiming arm 32. Also, the head 410 of the alignment tool 40 may have a recess (not shown) with two planar surfaces that can engage the planar surface 223 on the distal end of the body portion 220 of the osteotomy nail 20.

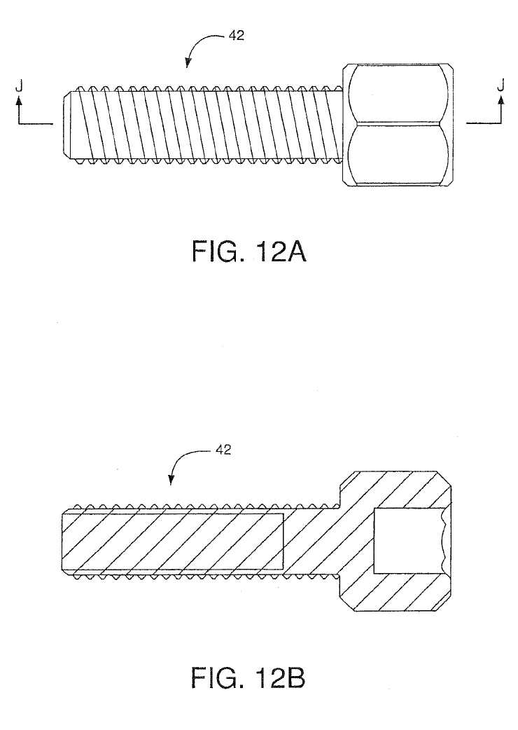

In one embodiment, as shown in FIGS. 12A-12B, the clamping fastener 42 is a bolt that can be inserted through the clamping hole 323 of the aiming arm 32 such that the collar hole 321 and slit 322 of the aiming arm 32 may be tightened. The clamping fastener, however, may be any other suitable fastener that can tighten the collar hole 321 and slit 322 of the aiming arm 32. Alternatively, a cam lock (not shown) may be used to tighten the collar hole 323.

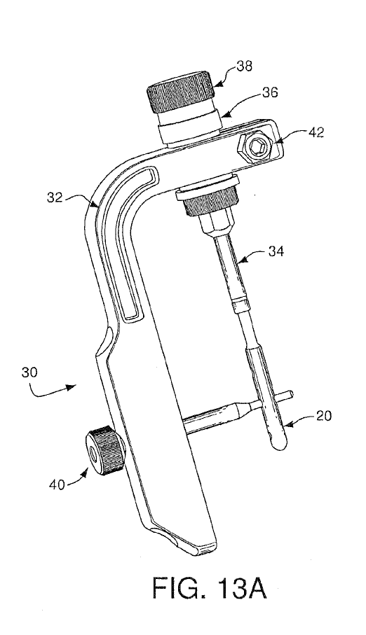

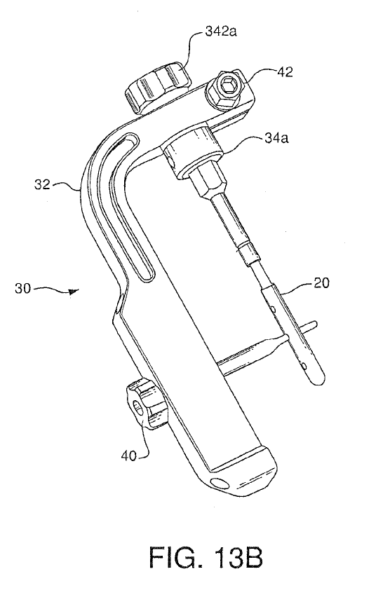

FIG. 13A illustrates one embodiment of an assembly of the osteotomy nail 20 and insertion device 30. The alignment tool 40 of the insertion device 30 provides a means for aligning the osteotomy nail 20 so that the osteotomy nail 20 may be held by the insertion device 30 in the proper orientation to accept the screws 23. The inserter tool 34, inserter collet 36, an inserter cap 38, and clamping fastener 42 of the insertion device 30 provide a means for holding the osteotomy nail 20 in proper alignment so that the osteotomy nail 20 does not lose proper alignment as it is being secured in the bone with the screw 23. The screw channels 331 of the insertion device 30 provide a means for guiding the insertion of the screws 23 into the osteotomy nail 20 to ensure proper alignment of the screws 23 with the screw bores 221 of the osteotomy nail 20. The inserter cap 38 and flange 342 of the inserter tool 34 provide a means for holding the inserter tool 34 so that the inserter tool's 34 axial movement in the collar hole 321 of the aiming arm 32 is restricted, The structures described above provide means for various functions, but these structures may be replaced or supplemented with other suitable structures for performing the same functions.

According to one embodiment, the assembly of the osteotomy nail 20 and insertion device 30 may be described with reference to FIG. 13A. The first step in the assembly is to secure the head portion 210 of the osteotomy nail 20 to the distal portion 340 of the inserter tool 34 using the fastening means 211, 343. The planar surfaces 223 on distal end of the body portion 220 of the osteotomy nail 20 can be engaged with the recess (not shown) on the head 410 of the alignment tool 40 to tighten the osteotomy nail 20 onto the inserter tool 34. Alternatively, a key or wrench may be used to engage the planar surfaces 223 on distal end of the body portion 220 of the osteotomy nail 20 and tighten the osteotomy nail 20 onto the inserter tool 34.

Preferably the inserter collet 36 is preassembled in the collar hole 321 of the aiming arm 32 by inserting the top end 361 of the inserter collet 36 into the collar hole 321 of the aiming arm 32 and securing the collet cap 366 onto the top end of the inserter collet 36. The collet cap 366 is attached to the top end 361 of the inserter collet 36 such that the inserter collet 36 is captured in the collar hole 321 of the aiming arm 32 by the flange 365 and collet cap 366. Preferably, attachment of the collet cap 366 does not restrict rotational or axial movement of the inserter collet 36 within the collar hole 321 of the aiming arm 32.

The next step is to insert the inserter tool 34 into the aiming arm 32. The proximal portion 341 of the inserter tool 34 is inserted into the bottom end 360 of the inserter collet 36. The complementary planar surfaces 346, 363 or polygonal structures 347, 364 of the inserter tool 34 and the inserter collet 36 allow the proximal portion 341 of the inserter tool 34 to be inserted inside the inserter collet 36 while preventing rotational movement of the inserter tool 34 with respect to the inserter collet 36. The inserter cap 38 is secured to the proximal portion 341 of the inserter tool 34 using the fastening means 348, 380. By securing the inserter cap 38, the inserter tool 34 is captured in the inserter collet 36 and collar hole 321 of the aiming arm 32 by the inserter cap 38 and the annular flange 342 of the inserter tool 34.

There are also alternative methods of inserting the inserter tool 34. For example, an inserter collet 36 without an annular flange 365 or a collet cap 366 may be used. Alternatively, the inserter collet 36 may be excluded altogether, and the inserter tool 34 may inserted directly into the collar hole 321 of the aiming arm 32.

FIG. 13B shows an alternative assembly of the osteotomy nail 20 and insertion device 30 incorporating the inserter tool 34a shown in FIG. 10A. As shown in FIG. 13B, the distal portion 340a of the inserter tool 34a may be attached to the head portion 210 of the osteotomy nail 20 and inserted through the collar hole 321 of the aiming arm 32. The inserter tool 34a has an annular flange 342a disposed around the proximal end 341a that stops and maintains the inserter tool 34a in the collar hole 321 of the aiming arm 32. Inserter tool 34 and inserter tool 34a can be used interchangeably with the osteotomy nail 20 and the insertion device 30. Reference to inserter tool 34 in the description following in this specification may refer to either inserter tool 34 or 34a unless expressly stated otherwise.

Once the inserter tool 34 is inserted into the aiming arm 32, the attached osteotomy nail 20 is aligned with respect to the aiming arm 32. The alignment tool 40 is inserted through the alignment channel 332 of the aiming arm 32 and into the alignment hole 222 of the osteotomy nail 20. The alignment tool 40 may be secured in the aiming arm 32 by engaging the fastening means 413 on the alignment tool 40 with the fastening means 333 in the alignment channel 332 of the aiming arm 32. The alignment tool aligns the alignment channel 332 of the aiming arm 32 and the alignment hole 222 of the osteotomy nail 20 such that the corresponding screw channels 331 of the aiming arm 32 and screw bores 221 of the osteotomy nail 20 are also aligned. For example, as depicted, the alignment channel 332 of the aiming arm 32 and the alignment hole 222 of the osteotomy nail 20 may be aligned such that the upper screw channel 331a targets the lower screw bore 221b and the lower screw channel 331b targets the upper screw bore 221b. However, other arrangements are possible, as appreciated by those skilled in the art in light of this disclosure.

Once the alignment hole 221 in the osteotomy nail 20 is aligned with the alignment channel 332 on the aiming arm 32, the inserter tool 34 holding the osteotomy nail 20 is secured with respect to the aiming arm 32. The inserter tool 34 may be secured in the aiming arm 32 by inserting a clamping fastener 42 through the clamping hole 323 of the aiming arm 32 and tightening the collar hole 321 and slit 322 of the aiming arm 32 around the inserter tool 34. However, it is possible to use any other means for tightening the collar hole 321 and slit 322 of the aiming aim 32 to prevent the combination of the inserter collet 36, inserter tool 34, and osteotomy nail 20 from moving with respect to the aiming arm 32.

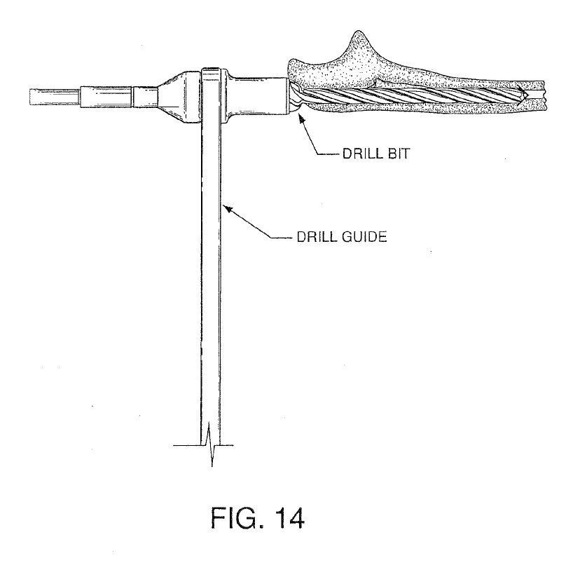

According to another aspect of the invention, the aiming arm 32 may be used to insert the osteotomy nail 20 into the upper end of the ulna to enable repair of an olecranon osteotomy. According to a preferred method, the osteotomy nail 20 is inserted and secured in the upper end of the ulna before the olecranon osteotomy is performed. FIGS. 14-17B illustrate one method of performing an olecranon osteotomy according to the present invention.

Referring to FIG. 14, a hole is drilled through the upper end of the ulna into the intramedullary canal to a depth adequate to accommodate the osteotomy nail 20 below the site of a proposed osteotomy. As depicted, the hole is preferably drilled with a drill bit having a shoulder portion that can create a countersink around the entrance of the hole. The countersink created by the drill bit allows the nail cap 24 to be at least partially countersunk when it is attached to the osteotomy nail 20.

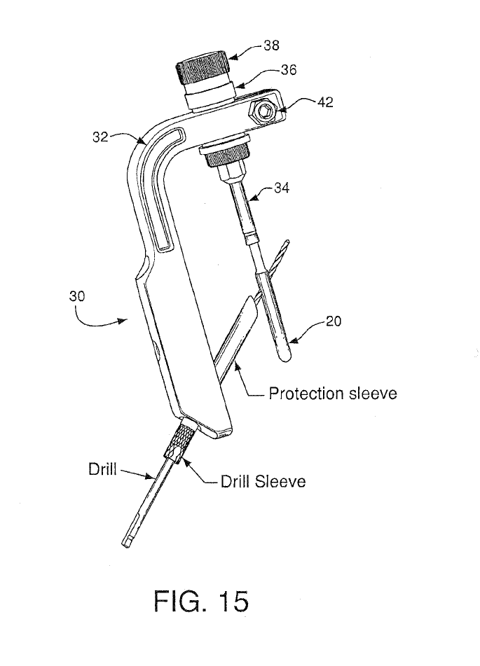

Referring back to FIGS. 13A-13B, the insertion device 30 is assembled and the osteotomy nail 20 is aligned and secured according the description detailed above. Now referring to FIG. 15, once the osteotomy nail 20 is aligned and secured, the alignment tool 40 can be removed and the osteotomy nail 20 may be inserted into the predrilled hole in the upper end of the ulna using the insertion device 30. As shown in FIG. 15, the aiming arm 32 of the insertion device 30 is disposed outside the bone with its screw channels 331 in alignment with the corresponding screw bores 221 of the osteotomy nail 20 disposed in the intramedullary canal of the bone. As shown in FIG. 15, a drill is passed through the lower screw channel 331b in the aiming arm 32, through one side of the bone, through the corresponding upper screw bore 221a in the osteotomy nail 20, and through the other side of the bone. Preferably, a drill sleeve and a protection sleeve are disposed in the lower screw channel 331b. The drill sleeve is disposed inside the protection sleeve, such that the drill is inserted through the drill sleeve and into the bone without causing damage to the surrounding soft tissue. This drilling process is repeated for the upper screw channel 331a and lower screw bore 221b.

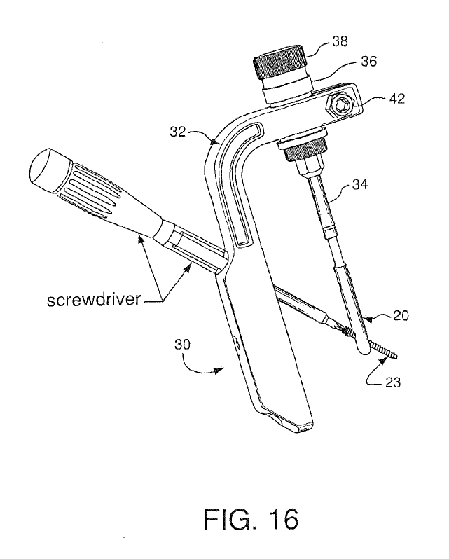

As shown in FIG. 16, the osteotomy nail 20 may be secured in the intramedullary canal of the bone by inserting a screw 23 through the upper screw channel 331a in the aiming arm 32, into one side of the bone, into the corresponding lower screw bore 221b in the osteotomy nail 20, and into the other side of the bone. The osteotomy nail 20 is preferably secured in the bone by driving a screw 23 through a screw bore 221 of the body portion 220 until the head portion 230 of the screw 23 contacts the body portion 220 of the osteotomy nail 20. Preferably, the screws 23 are driven with a screwdriver having a stop that prevents the screws 23 from being driven once the head portions 230 of the screws 23 contact the body portion 220 of the osteotomy nail 20. This screwing process is repeated for the lower screw channel 331b and upper screw bore 221a.

The combination of having the threaded head portions 230 of screws 23 contact the body portion 220 of the osteotomy nail 20 and having screw bores 221 disposed at opposing angles limits longitudinal, lateral, and rotational movement of the osteotomy nail 20 in the bone. The screw bores 221 in the osteotomy nail 20 are set at opposing angles oblique to the longitudinal axis (y) of the osteotomy nail 20 to limit the amount of longitudinal and rotational movement of the osteotomy nail 20 in the bone. Lateral and rotational movement of the osteotomy nail 20 in the bone due to clearance between the screws 23 and the screw bores 221 is limited by having the threaded head portions 230 of the screws 23 contact the body portion 220 of the osteotomy nail 20.

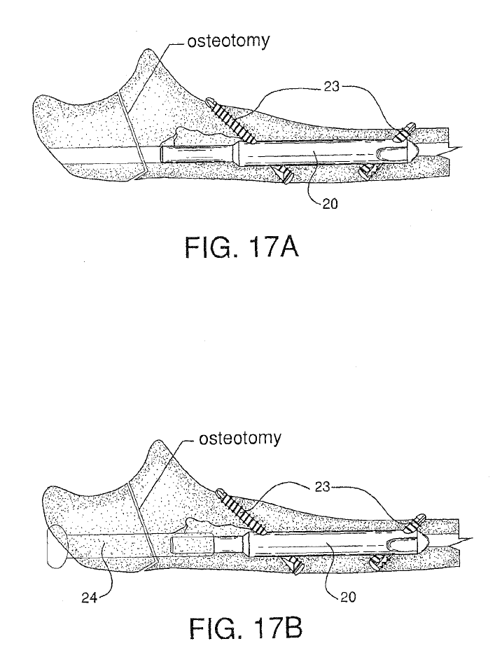

Once the osteotomy nail 20 is secured in the intramedullary canal of the ulna, the insertion device 30 may be disassembled and detached from the osteotomy nail 20 by, for example: removing the inserter cap 38 from the inserter tool 34, releasing the clamping fastener 323, removing inserter tool 34 from the aiming arm 32, and removing the distal portion 340 the inserter tool 34 from the osteotomy nail 20. Then, an olecranon osteotomy may be performed above (i.e. proximal from) the location of the osteotomy nail 20 as shown in FIG. 17A. Thereafter, the sectioned process of the bone may be replaced and the nail cap 24 may be inserted through the hole in the sectioned process of the bone and secured to the head portion 210 of the osteotomy nail 20 by using the fastening means 211, 242. The nail cap 24 provides compression and allows proper reduction of the olecranon osteotomy. Further, the nail cap 24 is at least partially countersunk into a countersink formed by a drill bit as described above. The countersinking of the nail cap 24 prevents the problem of prominence under the skin.

According to another aspect of the invention, the nail system 10 and insertion device 30 may be used to repair an olecranon fracture. As may be readily apparent to one skilled in the art, the insertion device 30, nail system 10, and method described herein may be applied to the reduction of an olecranon fracture in substantially the same manner that they are applied to the reduction of an olecranon osteotomy. For example, the osteotomy nail 20 may be inserted and secured in the intramedullary canal of a bone having a fractured olecranon by using the insertion device 30 in the same manner as described above. Then, the fractured olecranon may be replaced and the nail cap 24 may be inserted through a hole drilled in the fractured olecranon and secured to the head portion 210 of the osteotomy nail 20 by using the fastening means 211, 242. The nail cap 24 provides compression and allows proper reduction of the olecranon fracture.

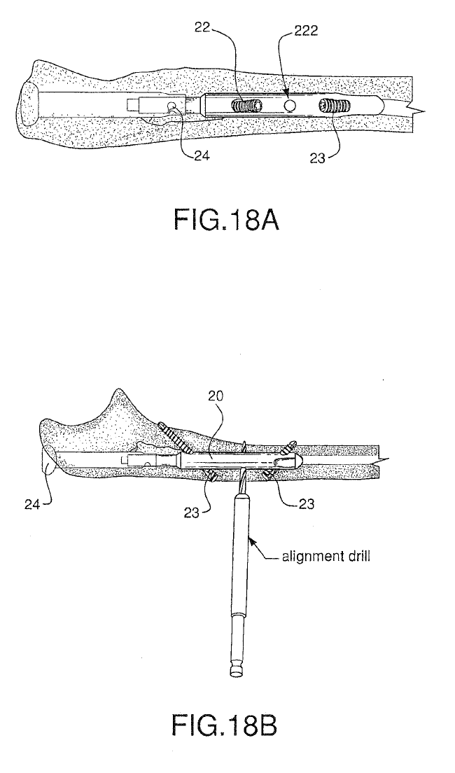

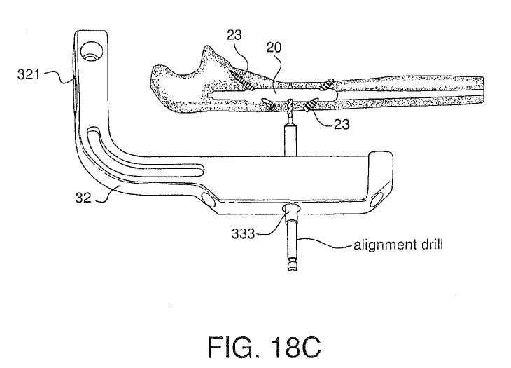

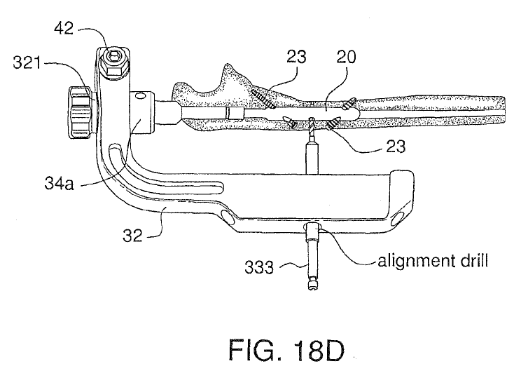

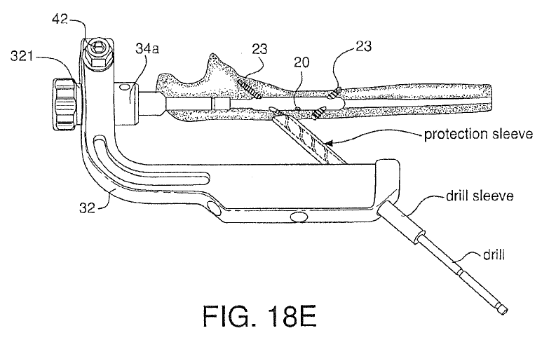

In accordance with another aspect of the invention, an exemplary method of removing an implanted nail system 10 will be described with reference to FIG. 18A-18E. FIG. 18A shows an osteotomy nail 20 attached to a nail cap 24 and secured into a bone by screws 23. As shown in FIG. 18B, an alignment drill may be used to drill through the bone and locate the alignment hole 222 of the osteotomy nail 20. The alignment drill is at least partially inserted into the alignment hole 222 of the osteotomy nail 20. Then, as shown in FIG. 18C, the nail cap 24 is removed from the osteotomy nail 20 and the aiming arm 32 is positioned over the alignment drill such that the alignment drill is disposed through the alignment channel 332 of the aiming arm 32. Thus, the alignment drill may be used to align the alignment channel 332 of the aiming arm 32 with the alignment hole 222 of the osteotomy nail 20. Then, as shown in FIG. 18D, the inserter tool 34a is inserted through the collar hole 321 of the aiming arm 32 and the distal end 340a of the inserter tool 34a is attached to the head portion 210 of the osteotomy nail 20.

When the alignment channel 332 of the aiming arm 32 and the alignment hole 222 of the osteotomy nail 20 are aligned with the alignment drill, and the inserter tool 34a is positioned through the collar hole 321 of the aiming arm 32 and attached to the osteotomy nail 30, the screw channels 332 of the aiming arm 32 and the screw holes 222 of the osteotomy nail 20 will be aligned. Once the screw channels 332 of the aiming arm 332 and the screw holes 222 of the osteotomy nail 20 are aligned, the inserter tool 34a attached to the osteotomy nail 20 may be secured in the aiming arm 32 by inserting a clamping fastener 42 through the clamping hole 323 of the aiming arm 32 and tightening the collar hole 321 and slit 322 of the aiming arm 32 around the inserter tool 34a. However, it is possible to use any other means for tightening the collar hole 321 and slit 322 of the aiming arm 32 to prevent the combination of the inserter tool 34a and osteotomy nail 20 from moving with respect to the aiming arm 32.

As shown in FIG. 18E, once the combination of the inserter tool 34a and osteotomy nail 20 is secured in the aiming arm 32, the alignment drill may be removed. Then, as shown in FIG. 18E, a drill may be passed through the lower screw channel 331b in the aiming arm 32 and through one side of the bone to expose the head 230 of a screw 23 in the corresponding upper screw bore 221a in the osteotomy nail 20. Preferably, a drill sleeve and a protection sleeve are disposed in the lower screw channel 331b. The drill sleeve is disposed inside the protection sleeve, such that the drill is inserted through the drill sleeve and into the bone without causing damage to the surrounding soft tissue. This drilling process is repeated for the upper screw channel 331a and screw 23 in the lower screw bore 221b. After, the heads of the screws 23 are exposed, the drill may be removed and a screw driver may be inserted through the upper 331a and lower 331b screw channels of the aiming arm 32 to remove the screws 23 in the upper 221a and lower 221b screw bores of the osteotomy nail 20.

Once the screws 23 are removed from the osteotomy nail 20, the osteotomy nail 20 may be removed from the bone. The osteotomy nail 20 may be removed from the bone by pulling on the aiming arm 32 that holds the inserter tool 34a attached to the osteotomy nail 20. Alternatively, the osteotomy nail 20 may be removed from the bone by loosening the collar hole 321 and slit 322 of the aiming arm 32 and pulling the inserter tool 34a attached to the osteotomy nail 20 out through the collar hole 321 of the aiming arm 32.

Thus, in accordance with the present invention, there is disclosed an osteotomy nail 20; a nail system 10 that includes the osteotomy nail 20, screws 23, and cap 24; and a nailing system that includes the nail system 10 and insertion device 30. The invention also includes a method for using the nail 20, the nail system 10, and the nailing system to repair an olecranon osteotomy or fracture.

1-27. (canceled)

28. An olecranon osteotomy nail system, comprising:
a nail including a body portion and a head portion, the body portion having a longitudinal axis and at least two screw bores spaced apart along the longitudinal axis and disposed oblique to the longitudinal axis at opposing angles relative thereto, the screw bores being disposed in a single plane bisecting the body portion along the longitudinal axis, the head portion located proximally of the body and having a nail fastening arrangement disposed on a surface thereof; and
a nail cap having a distal portion and a proximal portion, the distal portion having a cap fastening arrangement disposed on an interior surface of a recess thereof that engages the nail fastening arrangement such that the nail cap receives the head portion in the recess and can be secured on the head portion of the nail and provide compression to reduce an olecranon fracture.

29. The nail system of claim 28, wherein the nail further includes an alignment hole extending at least partially into the body portion, the alignment hole having a central axis generally transverse to the longitudinal axis of the body portion.

30. The nail system of claim 28, further comprising at least two screws being inserted through the screw bores.

31. The nail system of claim 28, wherein the screw bores are disposed oblique to the longitudinal axis at opposing angles between 30-60 degrees from the longitudinal axis.

32. The nail system of claim 28, further comprising a securing mechanism preventing the nail fastening arrangement from disengaging from the cap fastening arrangement.

33. The nail system of claim 32, wherein the securing mechanism includes a segment of the distal portion of the nail cap that is partially cut out and cantilevered to create a frictional force between the nail cap and the nail.

34. The nail system of claim 28, wherein the nail fastening arrangement and the cap fastening mechanism include threads.

35. The nail system of claim 28, wherein the distal portion of the nail cap has a first diameter and the proximal portion of the nail cap has a second diameter greater than the first diameter, the proximal portion sized and shaped to engage an exterior surface of a bone to provide the compression.

36. The nail system of claim 35, wherein the proximal portion of the nail cap is sized to engage a countersink on the exterior surface of the bone.

37. An olecranon osteotomy nail system, comprising:
a nail including a body portion and a head portion, the body portion having a longitudinal axis and having at least two screw bores spaced apart along a longitudinal axis and disposed oblique to the longitudinal axis, the screw bores being disposed in a single plane bisecting the body portion along the longitudinal axis, the head portion being located proximally from the body portion;
at least two screws disposed in the screw bores of the nail, each screw having a threaded head at a first end and a threaded body extending therefrom;
a nail fastening arrangement disposed on a surface of the head portion of the nail; and
a nail cap having a distal portion and a proximal portion, the distal portion having cap fastening arrangement disposed on an interior surface of a recess thereof engaging the nail fastening means such that the nail cap receives the head portion in the recess and can be secured on the head portion of the nail and provide compression to reduce an olecranon fracture.

38. The nail system of claim 37, wherein the screw bores are disposed at opposing angles relative to the longitudinal axis.

39. The nail system of claim 37, wherein the screw bores are disposed at parallel angles.

40. The nail system of claim 37, wherein the distal portion of the nail cap has a first diameter and the proximal portion of the nail cap has a second diameter greater than the first diameter, the proximal portion sized and shaped to engage an exterior surface of a bone to provide the compression.

41. The nail system of claim 40, wherein the proximal portion of the nail cap is sized to engage a countersink on the exterior surface of the bone.

42. The nail system of claim 37, wherein the nail fastening arrangement and the cap fastening mechanism include threads.
